(19) United States
(12) Patent Application Publication
(10) Pub. No.: US 2019/0119001 A1
(43) Pub. Date: Apr. 25, 2019

(54) POST DENTAL OR MEDICAL PROCEDURE PERSONAL CARE KIT

(71) Applicant: Jacob BERGER, Lakewood Ranch, FL (US)

(72) Inventor: Jacob BERGER, Lakewood Ranch, FL (US)

(21) Appl. No.: 16/013,653

(22) Filed: Jun. 20, 2018

Related U.S. Application Data
(60) Provisional application No. 62/575,241, filed on Oct. 20, 2017.

(51) Int. Cl. B65D 25/04; B65D 25/20; B65D 25/28; B65D 43/20; B65D 51/28; A61F 17/00

(52) U.S. Cl. CPC B65D 25/04; B65D 25/205; A61F 17/00; B65D 43/20; B65D 51/28; B65D 25/28

(57) ABSTRACT

A post-medical procedure personal care kit that includes a housing receptacle having a base defining an interior space, the base having an outer surface and an inner surface, and a lid movable with respect to the base. The lid includes an outer surface and an inner surface, the inner surface of the lid including a reflective material to enable a patient to see a reflective image of themselves, and a pouch containing at least one biohazard bag. The personal care kit includes an insert sized to fit within the interior space of the receptacle base, the insert comprising at least one interior compartment, where each of the interior compartments are configured to retain at least one item, where each of the items are received by a corresponding interior compartment in the insert, and each of the items relate to post-medical procedure care.

### CROSS-REFERENCE TO RELATED APPLICATION

This application claims the benefit of U.S. Provisional Patent Application No. 62/575,241, filed on Oct. 20, 2017, which is incorporated herein by reference in its entirety.

### FIELD

The present disclosure generally relates to dental or medical patient care products and more specifically to a kit that contains the necessary products and instructions that a dental or medical patient needs in order to provide self-care after a dental or medical procedure.

### INTRODUCTION

Often, after a medical or dental procedure, home care by the patient is recommended for better overall health and recovery and to decrease post-operative complications (such as, for example, delayed healing, pain, infection, etc.) or is the result of a choice by the patient who may have difficulties going to a post-op medical facility or physical therapy clinic for post-operative care either due to the financial issues involved or due to difficulties in the patient's schedule, i.e., school, work, family obligations. In many cases, home care is recommended. Home care is the care obtained by the patient once they are no longer in the immediate care of a medical or dental professional.

However, in many cases, improper or inefficient home care leads to complications after a medical or a dental procedure, such as infections, pain, and/or inflammation. The cause of the improper or inefficient home care is typically due to the insufficient understanding of what it takes to properly care for a dental procedure post operatively and what items and steps may help prevent complications and pain. In addition, there is a need to decrease the number of opioid prescriptions written per year due to the number of opioid related deaths and the number of individuals abusing the use of opioids after becoming addicts following a procedure that causes pain.

In addition, patients who select home care after a medical or dental procedure must follow specific instructions and obtain specific medical or dental post-operative items in order to administer proper home care. Often, the items that are needed are difficult or prohibitively expensive to obtain. In other cases, the patient simply does not possess the required products, which makes effective home care difficult and often inefficient. An effective alternative is required.

### SUMMARY

Many individuals have procedures that may require after-care. The present disclosure provides a post-medical procedure or post-dental personal care kit to assist patients in treating their inflammation, pain, and discomfort, and caring for their procedure site after they are no longer in the immediate care of a medical or dental professional. The personal care kit of the present disclosure conveys detailed post-operative care instructions to the patient and provides key aftercare items, such as, but not limited to, ibuprofen, acetaminophen, ice packs, tea bags, and hand sanitizer to be used as a hemostatic agent. The personal care kit also provides a safe way of disposing of soiled or blood-filled contents such as gauze, wound dressing, or sutures. The personal care kit may also contain Bacitracin or an equivalent preparation as an antimicrobial agent for topical application when appropriate. The personal care kit assists in reducing the amount of post-operative complaints of pain, infections, and complications that are a result of the gap of communication between patients and their doctors. The personal care kit offers an alternative to addictive medications, such as opioids, that are commonly prescribed after a dental or medical procedure and can result in addictions and overdoses. The present disclosure affords users all of the recommended items needed to care for a site post operatively that they may not otherwise have convenient access to or be able to afford.

According to one aspect of the disclosure, a post-medical procedure personal care kit is provided. The personal care kit includes a housing receptacle having a base defining an interior space, the base having an outer surface and an inner surface, and a lid movable with respect to the base. The lid includes an outer surface and an inner surface, the inner surface of the lid including a reflective material to enable a patient to see a reflective image of themselves, and a pouch containing at least one biohazard bag or equivalent disposal bag. The personal care kit also includes an insert sized to fit within the interior space of the receptacle base, the insert comprising at least one interior compartment, each of the at least one interior compartment configured to retain at least one item, and at least one item, each of the at least one item being received by a corresponding interior compartment in the insert, each of the at least one item related to post-medical procedure care.

In one embodiment of this aspect, the personal care kit further includes instructions including on how to use the at least one item. In another embodiment, the personal care kit further includes instructions that provide post-operative guidelines. In another embodiment, the personal care kit further includes instructions for allowing the patient to access at least one of an educational website and a video library containing information to assist the patient in post-medical procedural care.

In another embodiment, the outer surface of the base includes indicia related to a type of medical procedure the patient is recovering from. In another embodiment, each of the at least one item relates to the type of medical procedure indicated by the indicia.

In another embodiment, at least one of the inner surface of the base, the outer surface of the base, the inner surface of the lid and the outer surface of the lid further comprises a sleeve configured to retain a business card. In another embodiment, the at least one item includes at least one of antibacterial hand sanitizer, gauze, aspirin, pain reliever, anti-inflammatory, tea bag, salt, a hemostatic agent, sterile water, container, ice pack, and hand sanitizer. In another embodiment, the personal care kit further includes a hook and loop fastener for releasably securing the lid to an opposite side of the base. In another embodiment, the personal care kit further includes a handle for transporting the personal care kit.

According to another aspect of the disclosure, a post-medical procedure personal care kit is provided. The personal care kit includes a base that includes an outer surface and an inner surface, and at least one interior compartment, each of the at least one interior compartment configured to retain at least one item, and a lid movable with respect to the base. The lid includes an inner surface, the inner surface of the lid including a reflective material to enable a patient to see a reflective image of themselves, and a pouch containing at least one biohazard bag, and at least one item, each of the at least one item being received by a corresponding interior compartment in the base, each of the at least one item related to post-medical procedure care.

In one embodiment of this aspect, the personal care kit further includes instructions on how to use the at least one item. In another embodiment, the personal care kit further includes instructions that provide post-operative guidelines. In another embodiment, the personal care kit further includes instructions for allowing the patient to access at least one of an educational website and a video library containing information to assist the patient in post-medical procedural care.

In another embodiment, the outer surface of the base includes indicia related to a type of medical procedure the patient is recovering from. In another embodiment, each of the at least one item relates to the type of medical procedure indicated by the indicia. In another embodiment, at least one of the inner surface of the base, the outer surface of the base, the inner surface of the lid and the outer surface of the lid further comprises a sleeve configured to retain a business card.

In another embodiment, the at least one item includes at least one of antibacterial hand sanitizer, gauze, aspirin, pain reliever, anti-inflammatory, tea bag, salt, a hemostatic agent, sterile water, container, ice pack, and hand sanitizer. In another embodiment, the personal car kit further includes a hook and loop fastener for releasably securing the lid to an opposite side of the base.

According to another aspect of the present disclosure, a post-medical procedure personal care kit is provided. The personal care kit includes a housing receptacle comprising a base defining an interior space, the base having an outer surface and an inner surface, and a lid movable with respect to the base. The lid includes an outer surface and an inner surface, the inner surface of the lid including a reflective material to enable a patient to see a reflective image of themselves, and a pouch containing at least one biohazard bag. The personal care kit further includes an insert sized to fit within the interior space of the receptacle base, the insert comprising at least one interior compartment, each of the at least one interior compartment configured to retain at least one item, and at least one item related to post-medical procedure care, each of the at least one item being received by a corresponding interior compartment in the insert. The at least one item includes antibacterial hand sanitizer, gauze, aspirin, pain reliever, anti-inflammatory, tea bag, salt, a hemostatic agent, sterile water, container, ice pack, and hand sanitizer. The personal care kit further includes instructions about how to use the at least one item, and indicia on the outer surface of the base, the indicia identifying a type of medical procedure the patient is recovering from, the at least one item relating to the type of medical procedure identified by the indicia.

### BRIEF DESCRIPTION OF THE DRAWINGS

A more complete understanding of the present embodiments, and the attendant advantages and features thereof, will be more readily understood by reference to the following detailed description when considered in conjunction with the accompanying drawings wherein:

FIG. 1A is a perspective view of the post medical procedure personal care kit of the present disclosure showing an insert portion with a plurality of compartments and the base portion;

FIG. 1B is a perspective view of an alternate embodiment of the post medical procedure personal care kit of the present disclosure;

FIG. 2 is a perspective view of an exemplary embodiment of the post medical procedure personal care kit of the present disclosure showing the insert portion within the base portion;

FIG. 3 is a top view of the post medical procedure personal care kit of the present disclosure showing various exemplary items with the compartments in the insert portion;

FIG. 4 is a perspective view of an alternate embodiment of the post medical procedure personal care kit of the present disclosure; and

FIG. 5 is a perspective view of the post medical procedure personal care kit of the present disclosure in a closed configuration showing a sleeve on the outer side of the lid to retain a business card or other item.

### DETAILED DESCRIPTION

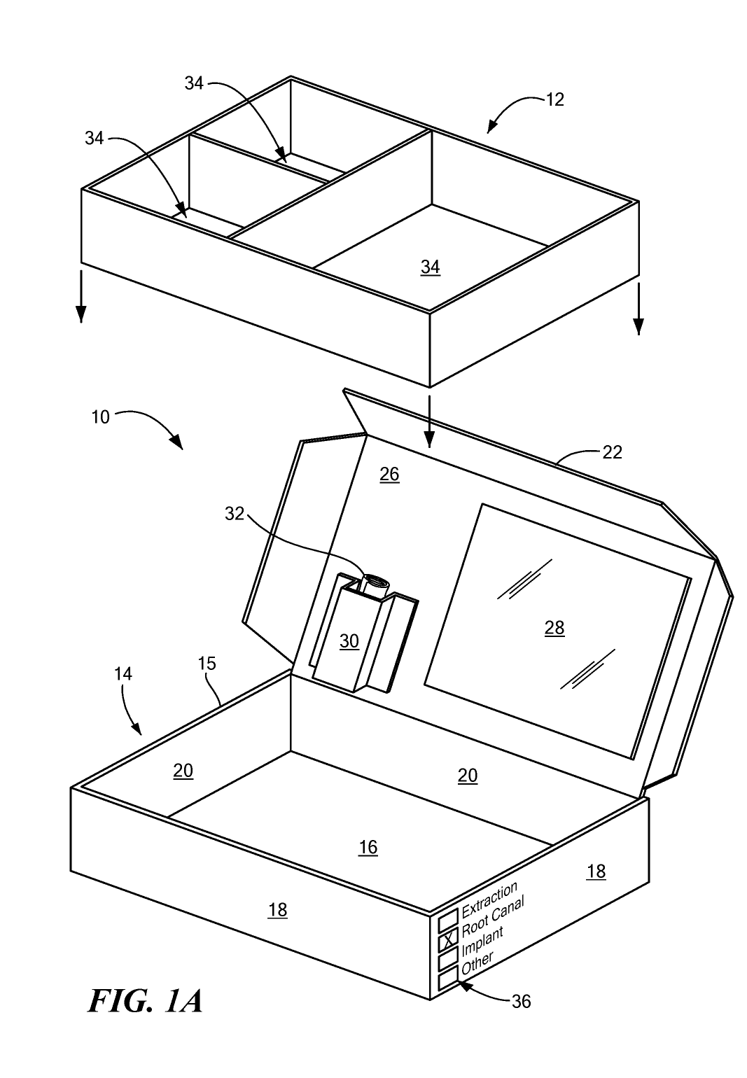

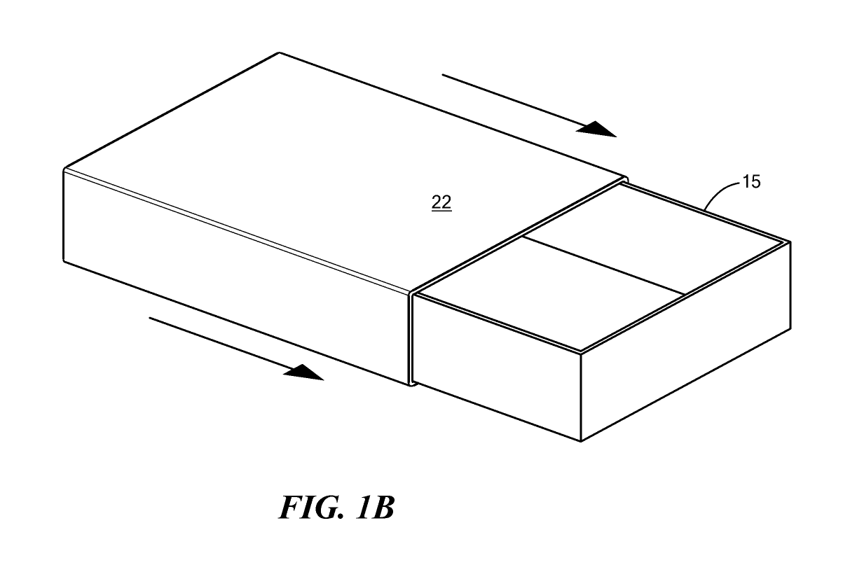

Referring now to FIGS. 1A-5, the post medical personal care kit (also referred to herein as “personal care kit”) of the present disclosure is designed to help users treat their inflammation and pain, and to provide care for the user after they are sent home from a medical facility or dental office after a procedure. As used herein, the term “medical procedure” shall encompass both medical and dental procedures and the term “post medical procedure personal care kit” or just “personal care kit” shall mean the personal care kit of the present disclosure which may contain either dental or medical items and be used by patients recovering from any type of medical or dental procedure. The items within the personal care kit relate to the type of medical or dental procedure that the patient is recovering from.

Referring now to FIG. 1A, the medical procedure personal care kit 10 of the present disclosure includes an insert 12, and a housing receptacle 14 such as a box. Housing receptacle 14 includes a base section 15 having an interior space 16, outer side surfaces 18, inner side surfaces 20, all of which forms an enclosure sized to receive insert 12. Personal care kit 10 can be of any dimensions or shape. For example, in one embodiment, personal care kit 10 has a height of between 1-1.5 inches, a length of between 4-6 inches, and a width of between 3.5-5.5 inches. Personal care kit 10 may be, for example, cuboid or rectangular in shape and its side walls, top and bottom, or lid may be of any thickness. It should be noted that the size, dimensions and shape of the personal care kit 10 may vary due to various reasons. For example, because certain medical procedures are more complex than others, kit 10 may need to be larger for these procedures to accommodate more items to account for the patient's recovery from a complicated and/or complex procedure.

In one embodiment, lid 22 of housing compartment 14 is rotationally affixed to one side of base 15 to allow the lid to be moved between an open and closed configuration, as shown in FIG. 1A. Lid 22 includes an outer surface 24 (not shown in FIG. 1A), inner surface 26, a mirror 28 or other type of reflective material to allow the patient to view themselves while using one or more items in personal care kit 10, and a receptacle or pouch 30 sized to retain one or more biohazard bags 32. Biohazard 32 may be any bag used to dispose of contents that may be soiled or that may contain blood. While a glass mirror 28 may be an option, in another embodiment, a platinum or silver sticker or sheet-like material may be used and applied to the inside surface 26 of lid 22. Therefore, although the term “mirror” or “reflective surface” is used in this disclosure, that term is not limited to a glass material commonly used for mirrors but may also encompass any type of reflective material. In another embodiment, mirror 28 may be located in an alternate place, i.e., not on the inside of lid 22 but in one of the compartments or at any other location in or on patient care kit 10.

In another embodiment, instead of lid 22 being rotationally affixed to one side of base 15 as shown in FIG. 1A, lid 22 can slide forward and backwards with respect to base 15, as shown in FIG. 1B. Thus, in this embodiment, a user can slide lid 22 either up or down with respect to base 15 in order to access the contents of kit 10.

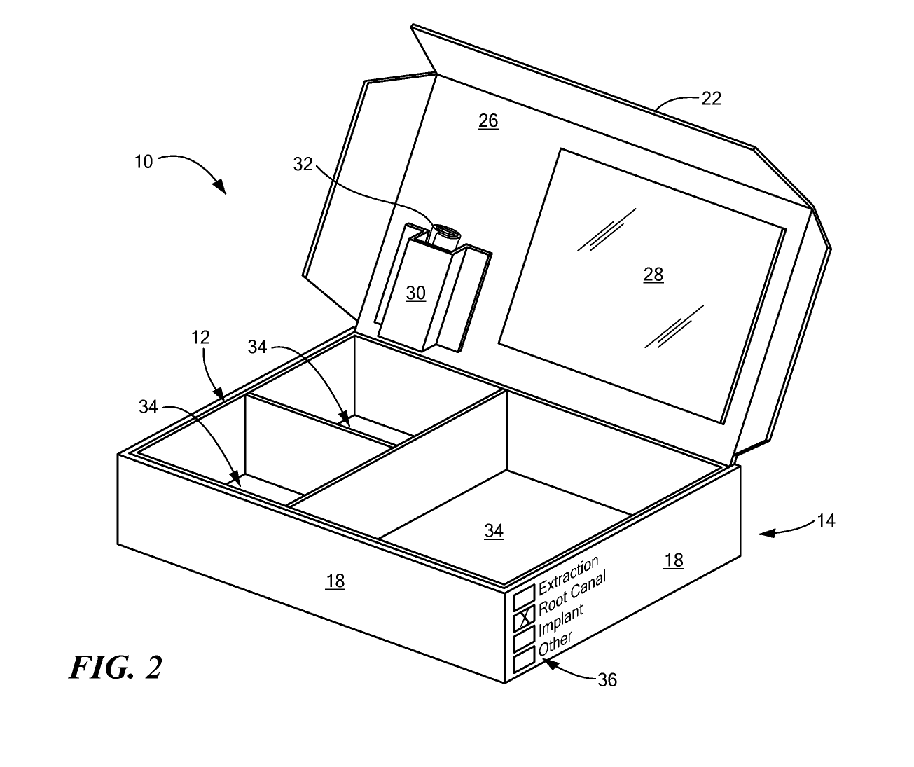

Prior to delivery to the patient, insert 12 is inserted within interior space 16 of housing receptacle 14, as shown in FIG. 2, and various items will be placed within compartments 34 in insert 12. Compartments 34 are each sized to retain one or more items that the patient may use while providing self-care after their procedure. These items will be discussed in greater detail below. Note that in an alternate embodiment, instead of personal care kit 10 comprising two separate components, i.e., insert 12 and housing receptacle 14, insert 12 and housing receptacle 14 may be one piece instead of two. Thus, for example, housing receptacle 14 may include compartments 34 within the interior space of housing receptacle 14.

In one embodiment, indicia 36 may be included on the outer surface 18 of base 15. Indicia 36 may include a listing of medical or dental procedures where one or more of these procedures are checked to indicate that the items contained within personal care kit 10 relate to the indicated medical procedure. This is advantageous because this allows for the kit 10 to be sealed and not have to be opened to observe the contents therein until first opened by the patient. Further, if kit 10 is being sold, for example, in a retail store, consumers can purchase the appropriate kit 10 knowing that the contents therein are recommended for that consumer's particular medical need by simply observing indicia 36. For example, in FIG. 2, the box for Root Canal is checked, indicating that this particular personal care kit 10 is for a patient that has recently undergone a root canal procedure and the items in personal care kit 10 relate to home-care for a patient that has undergone a root canal procedure.

In one embodiment, mirror 28, or a reflective sticker sheet is applied to the inside surface 26 of lid 22 of housing receptacle 14. This will allow the patient a convenient reflective image when the patient needs to utilize one or more items or to undertake specific post-procedure tasks, as will be discussed in further detail below. In another embodiment, the inner surface 26 of lid 22 of housing receptacle 14 includes a pouch 30 sized to contain one or more biohazard bags 32 to provide the user with the ability to discard all bloody items in the biohazard bag 32. The personal care kit 10 may also include instructions (not shown) to instruct the patient how to use the various items contained within personal care kit 10. A patient care website may be made accessible to the patient to provide accurate information directed to patients/users from doctors using information that is backed with peer-reviewed science.

A step-by-step instructional guide is included to show patients how to use the contained items, as well as specific rules and guidelines for optional post-operative care. The rules and guidelines are specifically tailored to the medical procedure that was performed on the patient and the contents in kit 10. In one embodiment, the guidelines may include instructions for the patient how to alternate the use of the medications contained in kit 10 for optimal effectiveness. In another embodiment, the guidelines may include a chart to track medication use.

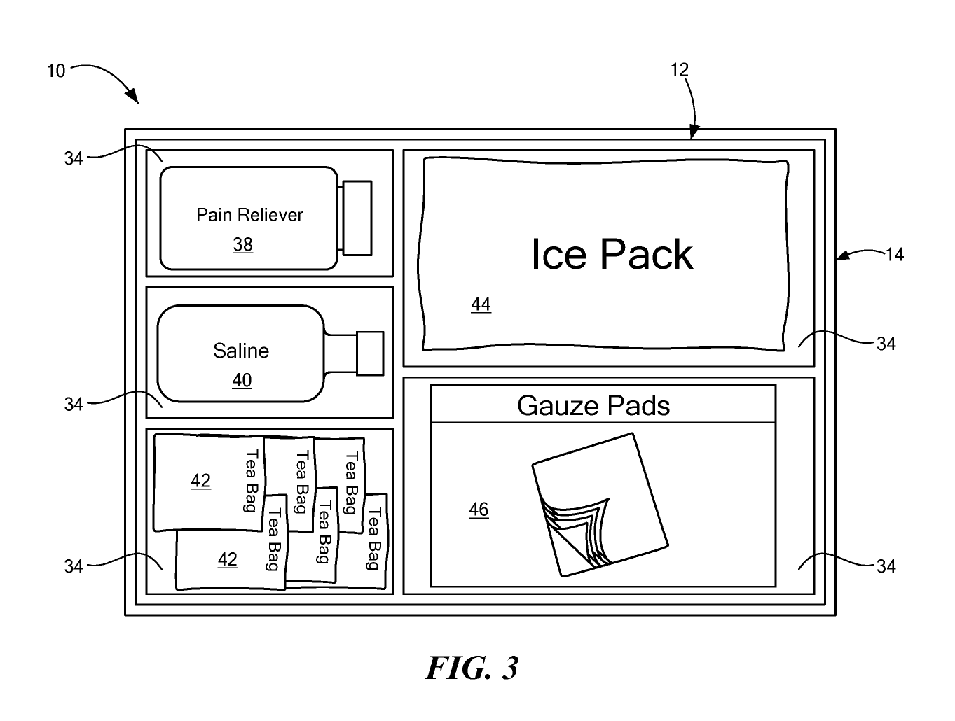

Referring to FIG. 3, each compartment 34 is sized to house one or more items of varying sizes and shapes. For example, the items may include a pain reliever 38 or nonsteroidal anti-inflammatory drugs (NSAIDs), an analgesic, ibuprofen, and/or acetaminophen. Other exemplary items may be, for example, saline solution 40, one or more tea bags 42 which contain black tea having tannic acid to help decrease bleeding, an ice pack 44, gauze pads 46, and hand sanitizer. It should be noted that the items shown in FIG. 3 are exemplary only. Other items relating to other types of specific medical or dental procedures may be included in the patient care kit 10. Further, there may be any number of compartments 34, i.e., more or less than the number of compartments 34 shown in the figures.

What follows is an exemplary use-case scenario for using the contents of the patient care kit 10. It should be noted that the instructions may direct the patient to use the following steps in different order, may direct the patient to use only some but not all of these steps, etc. Thus, the steps that follow represent only an exemplary use and the present disclosure shall not be limited to only the following steps in the following order.

In one embodiment, in a first step, the patient opens patient care kit 10 immediately after the medical procedure or within an allotted time, for example, within 20 minutes of a procedure. In other scenarios, patient care kit 10 can be opened and the instructions read prior to the medical procedure if desired. In this fashion, the patient will already know how to use the items once the medical or dental procedure has concluded and the patient is ready to use the patient care kit 10. In a second step, the patient reads the instructions (in the appropriate language) within the patient care kit 10 and/or the patient visits an interactive website that is listed within the patient care kit 10 or on the instructions or provided to the patient in some other manner where the patient can view videos and illustrations and obtain additional information about the contents of patient care lit 10 and how to use each item, if needed.

In a third step, the patient follows the instructions that are provided in the patient care kit 10. In one embodiment, the instructions may include directions regarding the avoidance of the certain events which will result in helping to decrease post-operative inflammation, pain, and/or complications. For example, if the patient is recovering from a dental procedure, the instructions may advise the patient not to smoke, not to drink through a straw, to avoid strenuous activities, to avoid laying completely flat for a given number of hours, i.e., 12 hours, after the dental procedure, to avoid eating sharp items that could cut the user's mouth such as, for example, chips, pretzels, peanuts, etc., and to avoid touching the operative are with non-disinfected hands.

In another scenario, if the procedure is one where bleeding may arise afterwards, the instructions may advise the user to bite on gauze pad 46 for a certain amount of time, for example, 20 minutes, in order to decrease bleeding and then advise the patient to discard the gauze pad 46 in biohazard bag 32 which can then be discarded safely. Patient care kit 10 includes a number of gauze pads 46, thus a new gauze pad 46 may be used to replace the old gauze pad 46 if needed. The instructions may also advise the patient that he or she use the included hand sanitizer before touching the wound site or any items that will contact the wound site. If the user is unable to replace gauze pad 46 with a new gauze pad 46 without the use of a mirror or reflective surface, patient care kit 10 conveniently includes mirror 28 to allow the user to more accurate switch from one gauze pad 46 to another and apply the new gauze pad 46 to the desired location. This step would not be needed if the user is not bleeding.

The instructions may direct the user/patient to take pain reliever 38 in a certain dosage, i.e., 400-600 mg of ibuprofen either immediately or within a specified time period after the procedure if medically able to do so depending on the user's medical history (i.e., a warning label may be included to give the patient an opportunity to read over for precautionary reasons). The instructions may then direct the patient to place ice pack 44 externally near the procedure location to help prevent and/or decrease swelling. Cold temperatures are known to decrease blood flow to the area, which in turn decreases the amount of inflammatory cells that travel to the location. The prevention of swelling and the ability to decrease swelling will lead to less pain experienced by the user. If bleeding persists, the user can bite down on tea bag 42 for a specified duration, i.e., for 15-20 minutes. Tea bags 42 contain black tea which has tannic acid, known to decrease bleeding. If the patient does not have access to a mirror to assist in placing tea bag 42, the user can utilize the mirror 28 built into the patient care kit 10, for example on the inner surface 26 of lid 22, to assist the user in a more accurate placement of the tea bag 42. Of course, apply tea bag 42 to the user's mouth may not be needed if no bleeding has occurred or if the procedure performed didn't result in a wound that bled.

The instructions may then direct the user to rinse gently with a salt (NaCl) water/saline (iodized salt and sterile water) solution 40 which is contained in a non-sterile plastic bottle included in patient care kit 10. The patient's use of the water/saline solution 40 may help soothe the area and decrease superficial edema (swelling) since it is by nature a weak anti-inflammatory combination of elements and is also considered to be a weak antimicrobial. The user will have the ability to reuse the plastic container for additional salt water rinses. In one embodiment, the plastic container containing the saline solution 40 is labeled to with markings to act as a guide to the user for an optimal salt-to-water ratio. The bottle can contain instructions on how to properly use the saline solution 40.

The instructions may direct the user to take a specific amount, i.e., 500 mg, of a different type of pain reliever, i.e., acetaminophen, if the patient is medically able (warnings may be included to allow the patient to make their own precautionary decision based on their medical history) at a specific time. For example, the instructions may direct the user to take the required dose of acetaminophen 3-5 hours following the ibuprofen dose taken in a previous step. In one embodiment, the patient may then be instructed to alternate between the ibuprofen and the acetaminophen every 3 to 5 hours until the medicine is used up or until pain no longer persists. A chart may be included within patient care kit 10 to assist the patient in monitoring and tracking their medication usage. In some embodiments, the items include a nonsteriodal anti-inflammatory drug (NSAID) and an analgesic (i.e., pain killer/antipyretic) and the user may be directed to take both of these items.

In one embodiment, the user can monitor their procedural wound if it was a procedure that included an incision and compare the wound to images contained on the website which all users will have access to, in order to better monitor their own health. Thus, the patient may consult the website and the pictures displayed thereon to observe how the proper healing of the wound should look like. This will act as a guide to educate the patient as to how they are making progress using the items in patient care kit 10. The user is also provided access to step-by-step illustrations and an online video library showing the patient how to use each item in the patient care kit 10 should the instructions within the package not be adequate or if there is a desire for a supplemental and/or more detailed set of explanations about how to use each of the items in patient care kit 10.

In one embodiment, patient care kit 10 is disposable following the use of the items therein. Some of the items may be kept and stored at the user's own risk for future use, however, it is recommend that a new patient care kit 10 be used for each medical or dental procedure. Items that may be kept, but at the users own risk, include but are not limited to, ice pack 44, remaining unused medication, etc.

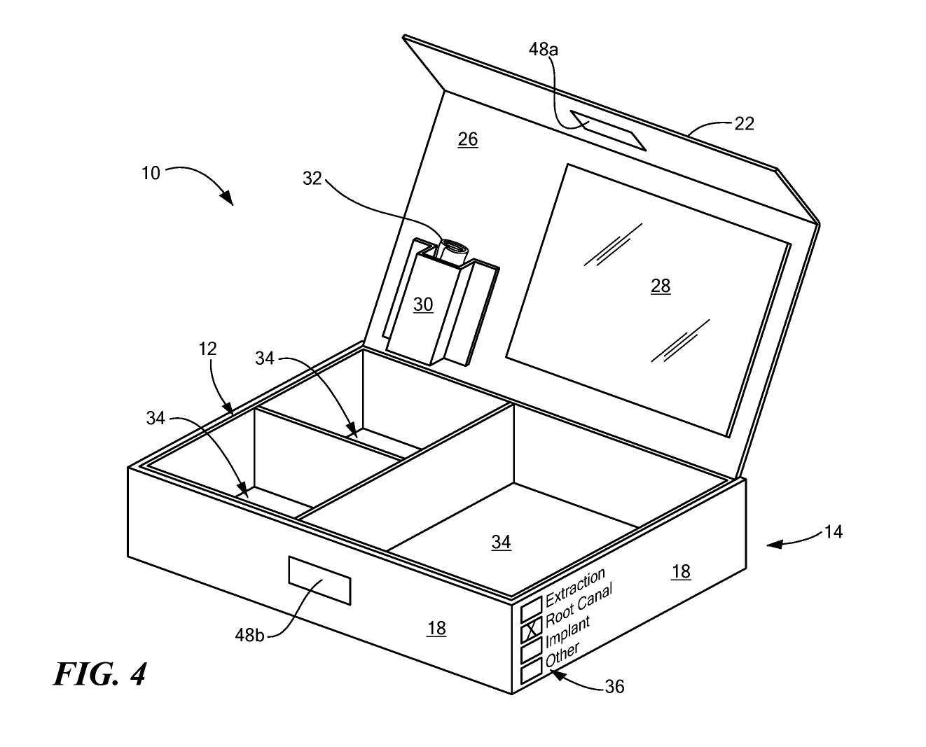

FIG. 4 illustrates an alternate embodiment of the patient care kit 10 of the present disclosure. In this embodiment, a pair of corresponding hook and loop fasteners 48a and 48b can be used to secure lid 22 closed upon base 15. For example, VELCRO® fasteners may be used, where one fastener 48a is affixed to the inside edge of a folded portion of lid 22 and mates with a corresponding fastener 48b affixed to an outer surface 28 of base 15 to releasably seal patient care kit 10 when not in use. In another embodiment, a handle (not shown) can be affixed to patient care kit 10 to allow the kit 10 to be transported.

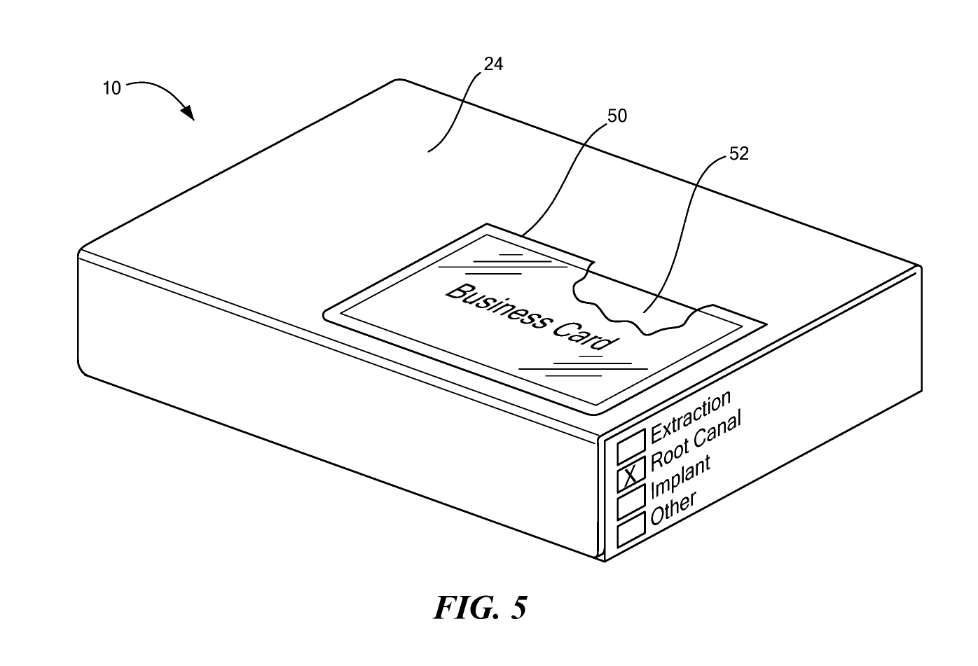

In another embodiment, shown in FIG. 5, a card sleeve 50 is affixed to the outer surface 24 of lid 22 and may be configured to receive and secure a business card 52 or other type of marketing or business material that may or may not contain contact information for a person or group of persons. In other embodiments, card sleeve 50 can be located on the inside surface of lid 22, or the outside or inside surface of the base 15. Further, instead of a card sleeve 50, advertisements can be affixed to any surface of the kit 10. For example, the outer surfaces of kit 10 may be used for marketing a business name and/or depict a logo. The outer surfaces of kit 10 may also include a sticker which contains a logo, business name, or any type of advertisement.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A post-medical procedure personal care kit comprising:
a housing receptacle comprising:
a base defining an interior space, the base having an outer surface and an inner surface; and
a lid movable with respect to the base, the lid comprising:
an outer surface and an inner surface, the inner surface of the lid including a reflective material to enable a patient to see a reflective image of themselves, and a pouch containing at least one biohazard bag;
an insert sized to fit within the interior space of the receptacle base, the insert comprising at least one interior compartment, each of the at least one interior compartment configured to retain at least one item; and
at least one item, each of the at least one item being received by a corresponding interior compartment in the insert, each of the at least one item related to post-medical procedure care.

2. The personal care kit of claim 1, further comprising instructions including how to use the at least one item.

3. The personal care kit of claim 1, further comprising instructions that provide post-operative guidelines.

4. The personal care kit of claim 1, further comprising instructions for allowing the patient to access at least one of an educational website and a video library containing information to assist the patient in post-medical procedural care.

5. The personal care kit of claim 1, wherein the outer surface of the base includes indicia related to a type of medical procedure the patient is recovering from.

6. The personal care kit of claim 5, wherein each of the at least one item relates to the type of medical procedure indicated by the indicia.

7. The personal care kit of claim 1, wherein at least one of the inner surface of the base, the outer surface of the base, the inner surface of the lid and the outer surface of the lid further comprises a sleeve configured to retain a business card.

8. The personal care kit of claim 1, wherein the at least one item includes at least one of antibacterial hand sanitizer, gauze, aspirin, pain reliever, anti-inflammatory, tea bag, salt, a hemostatic agent, sterile water, container, ice pack, and hand sanitizer.

9. The personal care kit of claim 1, further comprising a hook and loop fastener for releasably securing the lid to an opposite side of the base.

10. The personal care kit of claim 1, further comprising a handle for transporting the personal care kit.

11. A post-medical procedure personal care kit comprising:
a base comprising:
an outer surface and an inner surface; and
at least one interior compartment, each of the at least one interior compartment configured to retain at least one item;
a lid movable with respect to the base, the lid comprising:
an inner surface, the inner surface of the lid including a reflective material to enable a patient to see a reflective image of themselves, and a pouch containing at least one biohazard bag; and
at least one item, each of the at least one item being received by a corresponding interior compartment in the base, each of the at least one item related to post-medical procedure care.

12. The personal care kit of claim 11, further comprising instructions how to use the at least one item.

13. The personal care kit of claim 11, further comprising instructions that provide post-operative guidelines.

14. The personal care kit of claim 11, further comprising instructions for allowing the patient to access at least one of an educational website and a video library containing information to assist the patient in post-medical procedural care.

15. The personal care kit of claim 11, wherein the outer surface of the base includes indicia related to a type of medical procedure the patient is recovering from.

16. The personal care kit of claim 15, wherein each of the at least one item relates to the type of medical procedure indicated by the indicia.

17. The personal care kit of claim 11, wherein at least one of the inner surface of the base, the outer surface of the base, the inner surface of the lid and the outer surface of the lid further comprises a sleeve configured to retain a business card.

18. The personal care kit of claim 11, wherein the at least one item includes at least one of antibacterial hand sanitizer, gauze, aspirin, pain reliever, anti-inflammatory, tea bag, salt, a hemostatic agent, sterile water, container, ice pack, and hand sanitizer.

19. The personal care kit of claim 11, further comprising a hook and loop fastener for releasably securing the lid to an opposite side of the base.

20. A post-medical procedure personal care kit comprising:
a housing receptacle comprising:
a base defining an interior space, the base having an outer surface and an inner surface; and
a lid movable with respect to the base, the lid comprising:
an outer surface and an inner surface, the inner surface of the lid including a reflective material to enable a patient to see a reflective image of themselves, and a pouch containing at least one biohazard bag;
an insert sized to fit within the interior space of the receptacle base, the insert comprising at least one interior compartment, each of the at least one interior compartment configured to retain at least one item;
at least one item related to post-medical procedure care, each of the at least one item being received by a corresponding interior compartment in the insert, the at least one item including antibacterial hand sanitizer, gauze, aspirin, pain reliever, anti-inflammatory, tea bag, salt, a hemostatic agent, sterile water, container, ice pack, and hand sanitizer;
instructions about how to use the at least one item; and
indicia on the outer surface of the base, the indicia identifying a type of medical procedure the patient is recovering from, the at least one item relating to the type of medical procedure identified by the indicia.
